(19) United States
(12) Patent Application Publication
(10) Pub. No.: US 2024/0101948 A1
(43) Pub. Date: Mar. 28, 2024

(54) 3D Bioprinter That Cures Hydrogel Via Visible Light

(71) Applicant: United States of America as Represented by The Secretary of the Army, Alexandria, VA (US)

(72) Inventors: Seung J. OH, Champaign, IL (US); Tanner J. Wood, Mahomet, IL (US)

(21) Appl. No.: 17/955,463

(22) Filed: Sep. 28, 2022

(51) Int. Cl. C12M 1/26; B33Y 10/00; B33Y 30/00; B33Y 70/00; C12M 1/12; C12N 11/098

(52) U.S. Cl. CPC C12M 33/04; B33Y 10/00; B33Y 30/00; B33Y 70/00; C12M 25/02; C12N 11/098; C12N 2537/10

(57) ABSTRACT

A 3D bioprinter having a visible light source to photocure biomaterial is disclosed. The 3D bioprinter prints visible light-curable biomaterial along with viable cells, and visible light photocures the biomaterial while maintaining cell viability. Visible light 3D bioprinter systems and methods of printing are further disclosed.

### GOVERNMENT INTEREST

The subject matter of this disclosure was made with support from the United States Army Corps of Engineers. The Government of the United States of America has certain rights in this disclosure.

### FIELD

The present disclosure relates to a three-dimensional (3D) bioprinter that prints a hydrogel material with a visible light attachment that hardens (photocures) the hydrogel using visible light and maintains cell viability.

### BACKGROUND

There are 3D bioprinters that cure hydrogel material via high energy ultraviolet (UV) light. However, one major drawback of using this system is that UV light can potentially damage cellular DNA. UV light can also produce reactive oxygen species which can cause oxidative damage to the DNA. Hydrogel materials that can be hardened by visible light have been produced; however, there is no 3D bioprinter for photocuring hydrogel material via the visible light range. Visible light photocuring enables next-generation material fabrication, including hydrogels containing live cells. A need exists for a 3D bioprinter that allows hydrogel curing via visible light.

### SUMMARY

The present disclosure relates to a 3D bioprinter that includes a base unit having a support member adapted for mounting at least one bioprinter toolhead. A bioprinter toolhead is secured to the support member and is capable of dispensing a bioink material. The bioprinter also includes an object platform, and the bioprinter toolhead and object platform are movable in relation to one another. The bioprinter further includes a light source that is capable of outputting visible light directed toward the object platform, and a drive mechanism arranged to control movement of the bioprinter toolhead.

The present disclosure also relates to a 3D bioprinter system that includes an embodiment of a 3D bioprinter as disclosed herein, a visible light curable biomaterial provided in a non-cured state and capable of being dispensed by the bioprinter toolhead, and a source of viable cells.

The present disclosure further relates to a method for 3D printing a visible light-curable biomaterial that includes providing an embodiment of a 3D bioprinter as disclosed herein and a visible light-curable biomaterial in a non-cured state, printing a desired amount of the non-cured biomaterial, and photocuring the printed non-cured biomaterial with visible light output from the light source.

Other features and advantages of the present disclosure will be apparent from the following description of the drawings and detailed description, which should not be construed as limiting the disclosure to the embodiments shown and described.

### BRIEF DESCRIPTION OF THE DRAWINGS

FIG. 1 is a schematic illustration of a 3D bioprinter according to an embodiment.

FIG. 2 is a schematic illustration of a 3D bioprinter toolhead according to an embodiment.

FIG. 3 is an isometric view of a drive mechanism controller according to an embodiment.

FIG. 4 is an isometric view of a bioprinter toolhead holder according to an embodiment.

FIG. 5 is a schematic view of a light source according to an embodiment.

FIG. 6 is an isometric view of an adjustable light source holder according to an embodiment.

FIG. 7 is a schematic view of an object platform according to an embodiment.

FIG. 8 is an isometric view of a Petri dish holder according to an embodiment.

FIG. 9A-9C depict the bioprinting process according to an embodiment. FIG. 9A, Direct-Ink-Writing (DIW) extrusion process of hydrogel bioink formulation and visible light curing (photocrosslinking) solidifying the 3D construct. FIG. 9B, bioprinted 3D pyramidal and parabolic constructs (CAD file inset) seeded with genetically engineered E. coli bacteria in 7.5 wt % 3-CP-T hydrogel formulation (scale bar 2.5 mm). FIG. 9C, graphic depiction of isopropyl β-D-1-thiogalactopyranoside (IPTG) induction of bacteria laden 3D construct resulting in green fluorescent protein (GFP) expression via plasmid promoter.

FIG. 10A-10C are photographs and CAD file images of hydrogel constructs containing viable E. coli bacteria. FIG. 10A, 3D parabolic structures of 3-CP-T and 3-CP-V hydrogel bioinks with encapsulated bacteria rendered in comparison to the CAD input file and demonstrating bacterial cell viability through GFP expression imaged in the presence of UV light (scale bar 2.5 mm). FIG. 10B, bioprinted 3D pear construct embedded with viable E. coli bacteria expressing GFP upon IPTG induction, imaged in the presence of UV light (scale bar 2 mm). FIG. 10C, confocal microscope images of 3-CP-T and 3-CP-V hydrogels (7.5 wt %) embedded with viable E. coli bacteria that express GFP upon IPTG induction at 24 h and 72 h after photocrosslinking.

### DETAILED DESCRIPTION

While the present disclosure will be described in conjunction with specific embodiments, the disclosure can be applied to a wide variety of applications, and the description herein is intended to cover alternatives, modifications, and equivalents within the spirit and scope of the disclosure and the claims. The description in the present disclosure should not be viewed as limiting or as setting forth the only embodiments of the disclosure, as the disclosure encompasses other embodiments not specifically recited herein. The present disclosure is directed toward all novel and non-obvious features and aspects of the various disclosed embodiments.

Various embodiments of the present disclosure relate to a 3D bioprinter that prints a visible light-curable biomaterial, such as a hydrogel. In various embodiments, the 3D bioprinter prints a visible light-curable hydrogel along with viable cells. The 3D bioprinter photocures the hydrogel using a visible light source, while maintaining cell viability.

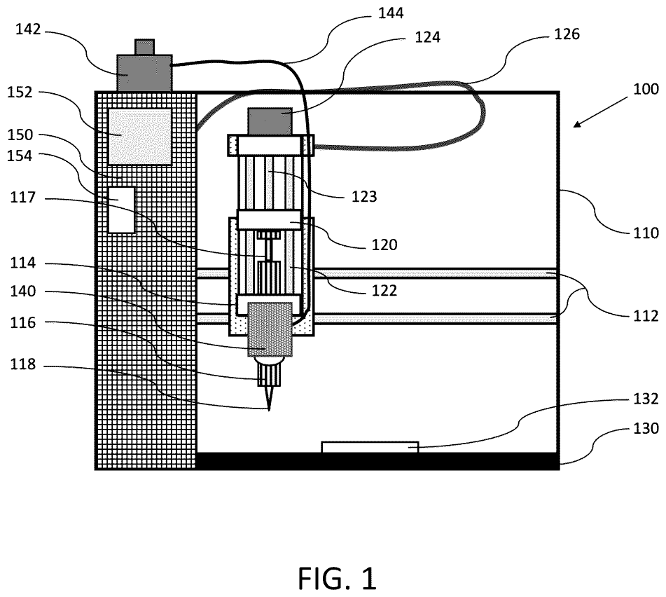

FIG. 1 illustrates an embodiment of the 3D bioprinter. The bioprinter 100 includes a base unit 110 that includes a support member 112 adapted for mounting at least one bioprinter toolhead 114, and at least one bioprinter toolhead 114 secured to the support member 112. In various embodiments, the bioprinter toolhead 114 is moveably secured to the support member 112 and allows for movement along a horizontal axis (X-axis), so that the toolhead 114 remains movable or slidable along the support member 112. In some embodiments, the bioprinter toolhead 114 is movable about the support member in the X-direction. In some embodiments, the bioprinter toolhead 114 is stationary. In some embodiments, the bioprinter toolhead 114 is moveable in other directions, such as the Z-direction. Embodiments of the support member 112 include a V-slot extrusion with grooves for wheels to move across, sliding rods that are stiff cylindrical bars that components can slide across, and linear rails that utilize a stiff track with small grooves for a carriage with ball-bearing spheres to fit on.

According to various embodiments, the bioprinter toolhead 114 is capable of dispensing a biomaterial or a bioink, such as a hydrogel. Embodiments of the biomaterial or bioink include hydrogels that are curable by visible light. In some embodiments, the biomaterial comprises, consists of, or consists essentially of 3-CP-T hydrogel, 3-CP-V hydrogel, or a combination thereof. These hydrogels are chemically synthesized three copolypeptide (CP) polymers (Threonine based (T) and Valine based (V)) that have visible light curing functionality.

In various embodiments, the bioprinter toolhead 114 comprises a precision volume dosing device or dispenser 116. In some embodiments, the bioprinter toolhead 114 dispenses the biomaterial by extrusion-based bioprinting. In some embodiments, the bioprinter toolhead 114 comprises a syringe pump extruder and various embodiments of the bioprinter toolhead 114 dispense the biomaterial by pneumatic, piston, or screw-based configurations. Embodiments of the toolhead 114 include a paddle 120 configured to push and/or pull a plunger 117 of the dispenser 116. Paddle 120 glides along one or more guide rail 122 in the Z-direction. Various embodiments include a worm screw 123 configured to move the paddle 120 up and/or down.

Various embodiments of the bioprinter toolhead 114 include a dosing device or extrusion element 118 with an outlet in a range of about 0.1 mm-5 mm, about 0.2 mm-4 mm, about 0.4 mm-2 mm, or about 0.5 mm-1 mm. In various embodiments, the extrusion element 118 includes a extrusion needle, such as a needle in a range of about 10-100 gauge, about 20-80 gauge, about 25-60 gauge, or about 14-27 gauge.

According to various embodiments, the bioprinter 100 includes a motor arrangement and a drive mechanism 124 to control movement of the bioprinter toolhead 114. In some embodiments, the drive mechanism 124 is connected to a control box 150 via one or more data cable 126.

According to various embodiments, the bioprinter 100 includes an print bed 130. In some embodiments, the print bed 130 is movable in the Y-direction. In some embodiments, the print bed 130 is stationary. In some embodiments, the print bed 130 is movable in other directions, such as the X-direction or the Z-direction. According to various embodiments, the print bed 130 and the bioprinter toolhead 114 are movable in relation to each other, such as in the X-, Y-, and/or Z-direction in relation to each other. According to various embodiments, the bioprinter 100 includes a motor arrangement and a drive mechanism to control movement of the print bed 130. In various embodiments, the bioprinter 100 is able to dispense biomaterial while moving in the X, Y, and Z directions.

According to various embodiments, the print bed 130 is configured with an object holder 132. According to various embodiments, the print bed 130 is configured to hold one or more of a Petri dish, a tissue culture dish, a multiwell plate, and a microtiter plate. In various embodiments, the object holder 132 is configured to hold a Petri dish or tissue culture dish, such as a 35 mm, 60 mm, 100 mm, or 150 mm diameter dish. In some embodiments, the object holder 132 is configured to hold a multiwell plate or microtiter plate, such as a 6 well, 12 well, 24 well, 48 well, or 96 well plate.

According to various embodiments, the bioprinter 100 includes a light source 140 that is capable of outputting visible light. In various embodiments, the visible light is directed toward the object platform 130 and the angle of the light source 140 is adjustable in relation to print bed 130.

According to various embodiments, the light source 140 provides a center wavelength (CWL) in a range of about 380 nm-750 nm, about 380 nm-500 nm, about 400 nm-420 nm, about 397 nm-413 nm, or about 400 nm-410 nm. In some embodiments, the light source 140 provides a CWL of about 405 nm. In various embodiments the CWL is 405 nm (+/−2 nm). Various embodiments of the light source 118 include a light emitting diode (LED). Some embodiments of the light source 140 include a bandpass filter to provide a desired wavelength.

Various embodiments of the bioprinter 100 include a light intensity controller 142 configured to control the light intensity of the light source 140. In some embodiments, the light intensity controller 142 is connected by wires 144 to the light source 140.

Various embodiments of the bioprinter 100 include a control box 150 that house various electronic components such as a controller board, communication interface, processing elements, cooling fans, and power supply. The control box 150 also includes an LCD screen 152 and power switch 154. In various embodiments, the bioprinter has a user interface arranged for user input and/or display of information.

Embodiments of the bioprinter 100 utilize a Cartesian-XZ-head motion system, also called the “i3-style”. The XZ-head motion system utilizes Z-axis rods that raise and lower an X-axis toolhead 114, where the toolhead 114 moves side to side in the X-axis. The toolhead therefore moves in the X and Z directions, while Y-axis rails or rods move the print bed backwards and forwards in the Y-axis. Exemplary embodiments of the bioprinter 100 utilize a LULZBOT® TAZ Workhorse or LULZBOT® TAZ Pro 3D Printer.

According to various embodiments, the bioprinter 100 runs off firmware that links the software to the hardware. The firmware converts inputs from software to an output that computer hardware can understand. The firmware “works out” the code from the software and accordingly gives an output to the stepper motors, heaters, display, etc. Exemplary embodiments of the bioprinter 100 utilize an open source Marlin firmware.

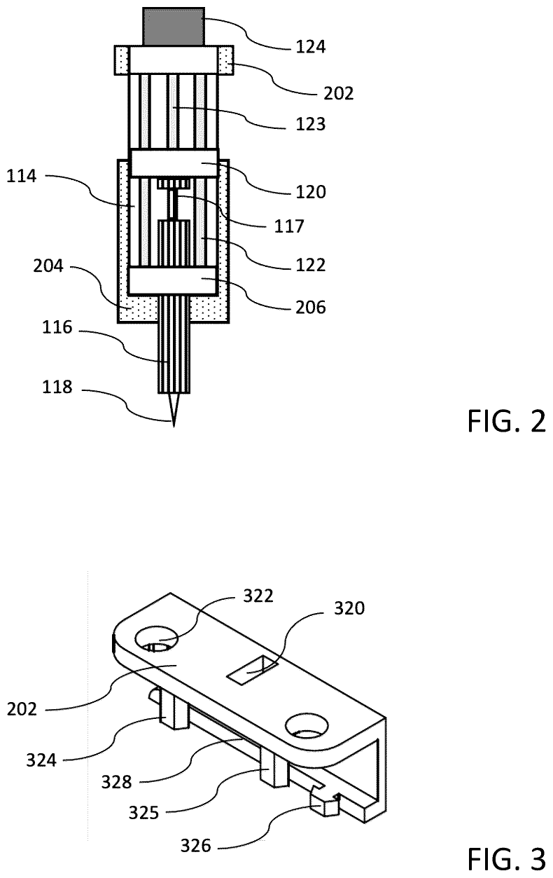

FIG. 2 illustrates a 3D bioprinter toolhead 114 and associated hardware according to an embodiment. Various embodiments of the bioprinter 100 include a cable management piece 202 configured to hold and secure one or more wire or cable to the bioprinter toolhead 114, such as wire or cable 126 and/or 144, and in some embodiments, to direct cables or wires out of the way of the printer build area. Various embodiments of the bioprinter 100 include a toolhead holder 204 configured to hold and secure the bioprinter toolhead 114. Some embodiments also include a dispenser clamp assembly 206 configured to secure the dispenser 116.

FIG. 3 shows an exemplary embodiment of the a cable management piece 202 that includes a slotted hole 320 to route wire or cable 126 and/or 144. Friction fittings 324, 325, and 326 are configured to slide into a t-slot on guide rails 122 to attach and secure the cable management piece 202. A slotted cavity 328 provides space and guidance for wires and/or cables. One or more holes 322 in the cable management piece 202 accommodate further attachment points for screws/bolts.

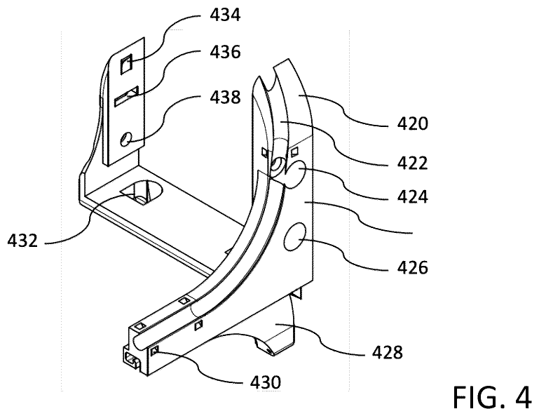

FIG. 4 shows an exemplary embodiment of bioprinter toolhead holder 204, having one or more holes 424, 426, 432, 438, to accommodate attachment points to the bioprinter toolhead 114. A slotted cavity 422 provides a guide to route light source wire 144 to the light source 116, and additional holes 430 provide a channel for zip ties meant to further secure light source wire 144. Bioprinter toolhead holder 204 also includes a feature 428 to mount an x-axis end stop, along with hole 434 to route end stop and leveling switch wire, and hole 436 for a zip tie meant to secure end stop and leveling switch wires.

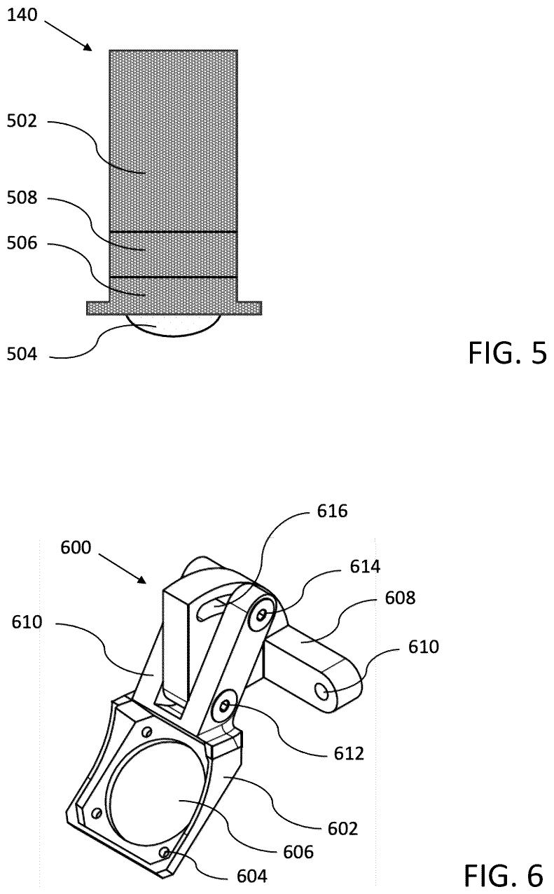

FIG. 5 shows an exemplary embodiment of a visible light source 140 assembly. In various embodiments, the light source assembly 140 includes an LED 502. Embodiments of the LED 502 include internal electronics and a heat sink. An exemplary LED is Thorlabs Model M405L4 or Model M405LP1 (New Jersey, USA) having an LED output power in a range of about 1000-1700 mW and a bandwidth (FWHM) of about 12-12.5 nm. Light source assembly 140 also includes a lens 504 and a mounting tube 506. In various embodiments, an extension tube 508 allows the lens 504 to be positioned to provide a proper amount of desired focus. In various embodiments, the light source assembly 140 also includes an optical bandpass filter to direct light frequencies within a certain desired wavelength range and block unwanted frequencies outside that range.

In various embodiments of the bioprinter 100, the position of the light source assembly 140 is adjustable. FIG. 6 shows an exemplary embodiment of an adjustable light source holder 600 configured to secure the light source assembly 140 and adjust the angle of the light source assembly 140 in relation to the print bed 130. Various embodiment of light source holder 600 includes a housing 602 for the light source assembly 140. Housing 602 includes holes 604 positioned for bolt attachments to mounting tube 506, and a hole 606 positioned for the lens 504 and to allow light from the LED to travel to the print bed 130.

Embodiments of the light source holder 600 have a mount 608 that allows the light source holder 600 to connect to the bioprinter 100. In some embodiments, mount 608 connects to the dispenser clamp assembly 206 with bolts through holes 610. Housing 602 connects to mount 608 via attachment arms 610. The light source holder 600 is adjustable in several directions via one or more pivot point 612 and 614 and slot 616. Various embodiments include bolts with wing nuts that tighten and secure the light source holder 600 and corresponding light source assembly 140 at a desired angle and position.

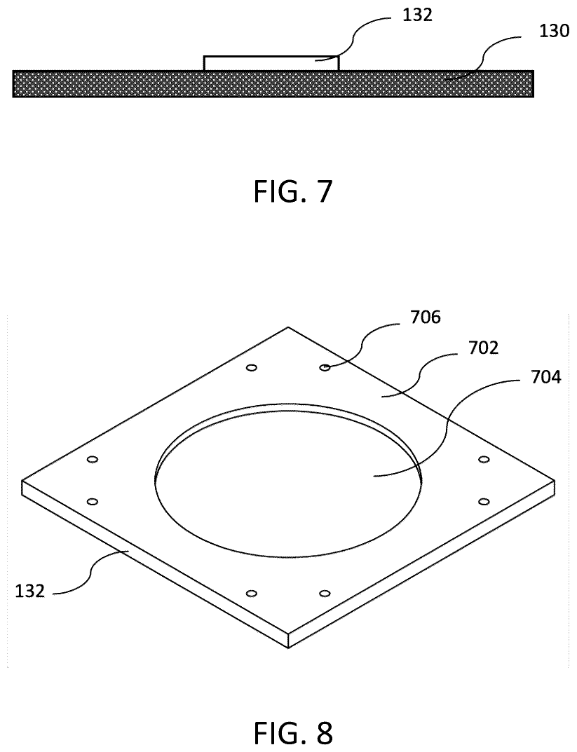

As shown in FIG. 7, in various embodiments of the bioprinter 100, the print bed 130 is configured with an object holder 132 to hold one or more of a Petri dish, a tissue culture dish, a multiwell plate, a microtiter plate, or a glass slide. FIG. 8 shows an exemplary embodiment of an object holder 132 configured to hold a Petri dish. In this exemplary embodiment, the object holder 132 includes a plate 702 having a friction fit hole 704 configured to hold a 100 mm petri dish. Plate 702 has one or more hole 706 for bolts to attach the plate 702 to the print bed 130. Various embodiments include one or more spring and wing bolt that allow one to level the plate.

Various embodiments of the present disclosure relate to a 3D bioprinter system. Embodiments of the system include an embodiment of a 3D bioprinter disclosed herein, a visible light-curable biomaterial provided in a non-cured state and capable of being dispensed by the bioprinter toolhead, and a source of viable cells.

According to various embodiments of the system, the biomaterial is a hydrogel that is photocurable by visible light. In some embodiments, the biomaterial comprises, consists of, or consists essentially of 3-CP-T hydrogel, 3-CP-V hydrogel, or a combination thereof. In various embodiments, the 3D bioprinter prints a visible light-curable hydrogel along with viable cells. The 3D bioprinter photocures the hydrogel using a visible light source, while maintaining cell viability.

According to various embodiments, the viable cells are plant cells, animal cells, insect cells, algae cells, yeast cells, fungus cells, bacteria cells, protozoa, microorganisms, or any combination thereof. According to various embodiments, the cells are viable in the biomaterial.

Various embodiments of the present disclosure relate to a method for 3D printing a visible light-curable biomaterial. Embodiments of the method include providing an embodiment of a 3D bioprinter disclosed herein, providing a visible light-curable biomaterial provided in a non-cured state, printing a desired amount of the non-cured biomaterial containing viable cells, and visible light photocuring the printed non-cured biomaterial with visible light output from the light source. In various embodiments, the visible light photocuring is initiated manually or automatically.

In various embodiments of the method, the biomaterial is a hydrogel that is curable by visible light. In various embodiments, the biomaterial is a fluid, semi-solid, or solid composition, or a combination thereof. In some embodiments, the biomaterial comprises, consists of, or consists essentially of 3-CP-T hydrogel, 3-CP-V hydrogel, or a combination thereof. In various embodiments, the 3D bioprinter prints a visible light-curable hydrogel containing viable cells. The 3D bioprinter photocures the hydrogel using a visible light source, while maintaining cell viability.

According to various embodiments of the method, various types of cells are printed. For example, both procaryotic and eucaryotic cells are printed. In various embodiments, the viable cells are plant cells, animal cells, insect cells, algae cells, yeast cells, fungus cells, bacteria cells, protozoa, microorganisms, or any combination thereof. According to various embodiments, the cells are viable in the biomaterial. In various embodiments, the cells remain viable after curing the biomaterial. In various embodiments, the method further includes culturing the cured biomaterial under appropriate culture conditions for the cells. In some embodiments, the non-cured biomaterial contains the viable cells. In some embodiments, the viable cells are provided separate from the biomaterial.

According to various embodiments, upon printing and photocuring the biomaterial containing viable cells, at least about 25% of the cells remain viable after incubation for 24 hours under appropriate culture conditions. For example, in various embodiments, at least about 25%, at least about 50%, at least about 75%, or at least about 85% of the printed cells remain viable in the cured biomaterial after incubation for 24 hours under appropriate culture conditions.

According to various embodiments of the method, the printed density is selectively varied to attain a desired result. The desired density generally depends on a variety of factors, such as the nature of the cells, the type of biomaterial, and the ultimate application for which the cells are intended. In some embodiments, the printed cells are relatively spaced apart (i.e., low cell density). In such cases, the cell density ranges from about 0.1 to about 2 cells per square millimeter. In other embodiments, the printed cells are in relative close proximity, (i.e., high cell density). In such cases, the cell density ranges from about 0.01 to about 2 cells per square micrometer.

### EXAMPLES

Viability and functionality of bacteria laden DIW printed 3D constructs. The rapid nature of hydrogel photocrosslinking using mild visible-light irradiation coupled with the hydrogel mechanical stability and inherent biodegradability prompted an investigation into their use as Direct Ink Writing (DIW) printable stationary matrix for bacteria. A genetically engineered Escherichia coli DH5α containing a pZEMB8 plasmid was used for the study, which was genetically responsive to isopropyl β-D-1-thiogalactopyranoside (IPTG) induction for the expression of green fluorescent protein (GFP).

Initially, both 3-CP-T and 3-CP-V copolypeptide hydrogels were screened to determine their effect on functionality of encapsulated E. coli at a fixed concentration (7.5 wt %). Upon visual examination, each hydrogels' physical appearance was uncompromised, as no discernible difference in turbidity was observed in 3-CP-T and 3-CP-V hydrogel inks with or without bacteria encapsulated. Both hydrogels were capable of supporting the cell function of embedded bacteria after short term photocuring (10 min) as GFP expression was observed after 24 h of incubation in an initial assay.

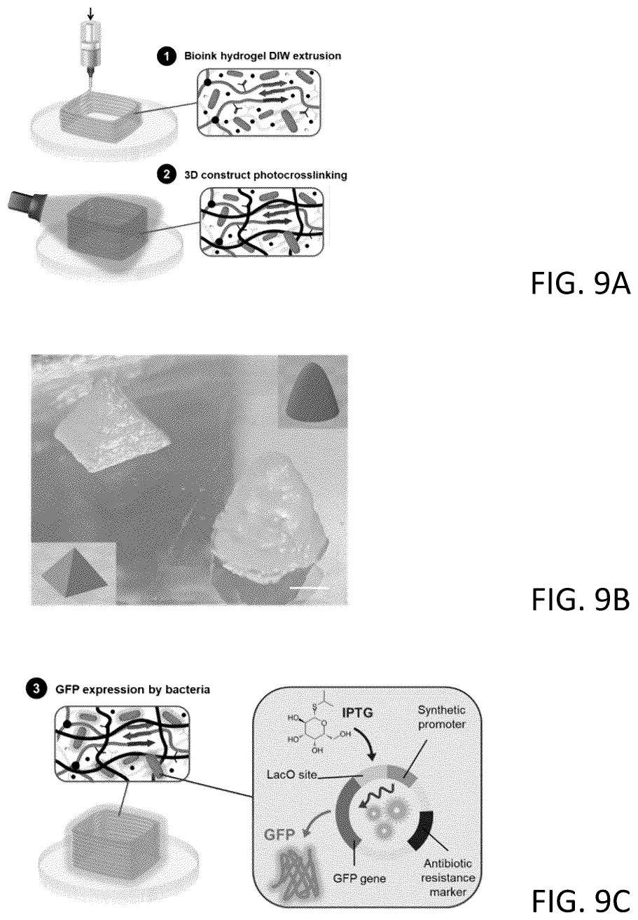

To fabricate “living” 3D hydrogel structures, a three-step procedure was conducted that supports both mechanical stability and cell functionality (FIG. 9A). Bacteria were first blended with each of the 3-CP-T and 3-CP-V copolypeptide hydrogel formulations (fixed at 7.5 wt % copolypeptide) to form the bioink, which was then DIW extruded to form contrasting geometric 3D shapes (FIG. 9B, scale bar 2.5 mm). Photocrosslinking with visible-light was then performed, followed by chemical induction with IPTG to promote GFP expression within the seeded bacteria. GFP expression was clearly observed using fluorescence microscopy, suggesting high viability and functionality of the embedded bacteria (FIG. 9C).

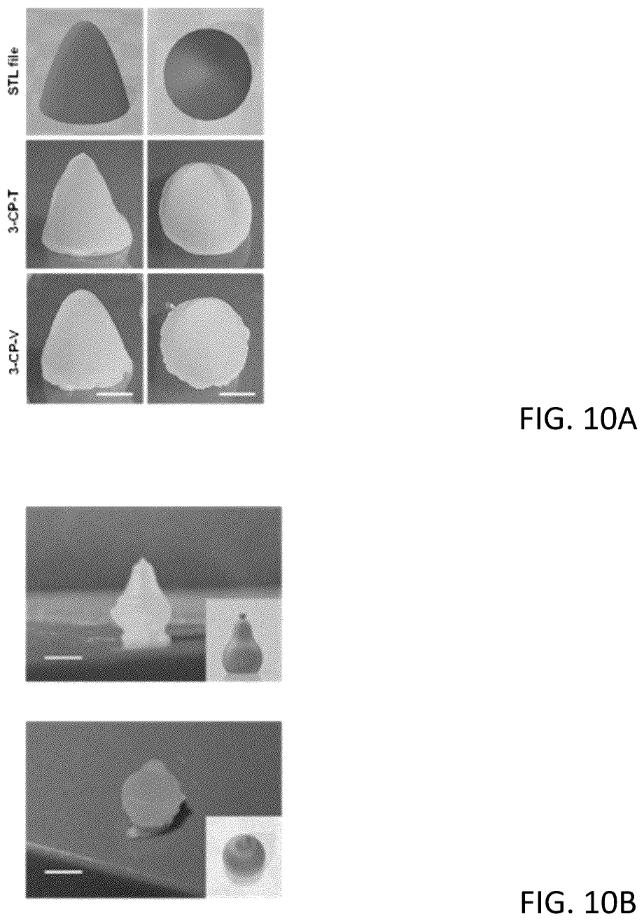

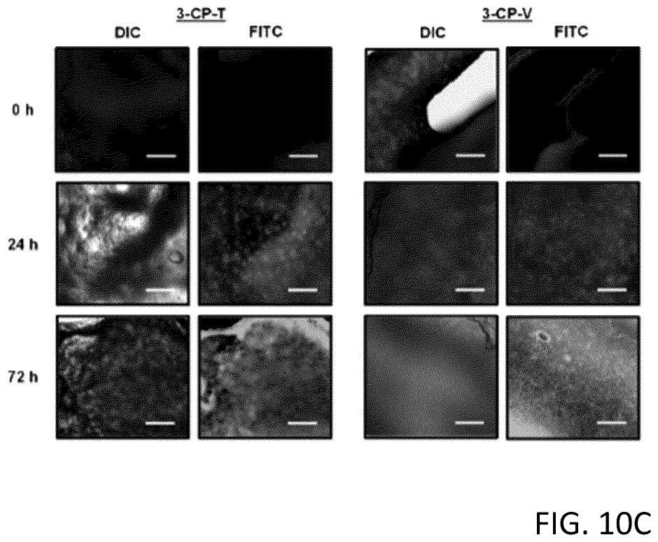

The 3-CP-T and 3-CP-V bioinks readily form structures with high resolution and print fidelity, accurately resembling the input CAD file for parabolic and pyramidal 3D structures. Cell viability and function of encapsulated bacteria was also maintained, as both 3-CP-T and 3-CP-V provided an inert support matrix for the E. coli colonies during printing and IPTG induced GFP expression upon UV exposure for visualization in both parabolic and pyramidal structures (FIG. 10A, scale bar 2.5 mm).

This occurred after photocrosslinking with visible light, which afforded highly stiff materials as observed rheologically. It appeared that the higher stiffness of 3-CP-V (2890 kPa) when compared to 3-CP-T (294 kPa) had no significant effect on the viability and function of embedded E. coli upon initial observation. A more complex 3D pear structure was also readily printed with good geometric fidelity showing similar fluorescence to other printed structures upon UV exposure, conveying the materials exceptional printability (FIG. 10B, scale bar 2 mm).

In order to examine the longer term stability and extension of bacterial growth, the functionality of embedded bacteria in 3-CP-T and 3-CP-V hydrogels was observed at different time intervals. GFP expression at 0 h, 24 h, and 72 h was examined using fluorescent microscopy post-photocrosslinking and IPTG induction. The photocrosslinked hydrogel bioink did not exhibit any fluorescence stemming from bacterial production at 0 h as a result of low GFP expression. This was in stark contrast to 24 h, when the number of GFP expressing bacteria significantly increased. This was observed for both 3-CP-T and 3-CP-V bioinks, with similar exponential bacterial growth and GFP expression, suggesting both hydrogels had similar biocompatibility and can facilitate proliferation of bacterial cells (FIG. 10C, scale bar 100 μm).

At 72 h, a distinct difference was observable with bacteria colonies within the 3-CP-T and 3-CP-V matrices. Colony size and aggregation was more pronounced within 3-CP-T, with differential interference contrast (DIC) images showing a higher exponential bacterial growth than 3-CP-V (FIG. 10C). Because 3-CP-T is less stiff when compared to 3-CP-V as observed rheologically, it could be postulated that the lower density matrix facilitates enhanced proliferation and bacterial colony agglomeration more readily. This demonstrates that bacterial cell growth and fate can be programmed over time.

It is to be understood that where the claims or specification refer to “a” or “an” element, such reference is not to be construed that there is only one of the element.

It is to be understood that where reference is made herein to a method or process that includes two or more defined steps, the defined steps can be carried out in any order or simultaneously (except where context excludes that possibility), and the process can also include one or more other step which are carried out before any of the defined steps, between two of the defined steps, or after all of the defined steps (except where context excludes that possibility). Methods of the disclosure may be implemented by performing or completing manually, automatically, or a combination thereof, selected steps or tasks.

For purposes of the disclosure, terms of approximation, such as “about,” should be interpreted according to their ordinary and customary meanings as used in the associated art unless indicated otherwise. Absent a specific definition and absent ordinary and customary usage in the associated art, such terms should be interpreted to be ±10% of the base value.

When a range is given as “(a first number) to (a second number)” or “(a first number)-(a second number)” this means a range whose lower limit is the first number and whose upper limit is the second number. For example, 25 to 100 or 25-100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc.

While inventive concepts have been described and illustrated herein by reference to certain embodiments, various changes and further modifications may be made by those of ordinary skill in the art without departing from the spirit of the inventive concept, the scope of which is to be determined by the following claims.

What is claimed is:

1. A three-dimensional (3D) bioprinter comprising:
a base unit comprising a support member adapted for mounting at least one bioprinter toolhead;
a bioprinter toolhead secured to the support member and capable of dispensing a biomaterial;
a print bed, the print bed and the bioprinter toolhead movable in relation to one another along an X-axis, a Y-axis, and a Z-axis;
a light source capable of outputting visible light directed toward the print bed; and
a drive mechanism arranged to control movement of the bioprinter toolhead.

2. The 3D bioprinter of claim 1, further comprising a light intensity controller configured to control the light intensity of the light source.

3. The 3D bioprinter of claim 1, wherein the print bed is configured to hold one or more of a Petri dish, a tissue culture dish, a multiwell plate, a microtiter plate, or a glass slide.

4. The 3D bioprinter of claim 1, wherein the print bed is configured to move in one or more of X, Y, and Z direction.

5. The 3D bioprinter of claim 1, wherein the bioprinter toolhead is configured to move in one or more of X, Y, and Z direction.

6. The 3D bioprinter of claim 1, wherein the light source provides a center wavelength (CWL) of about 405 nm.

7. The 3D bioprinter of claim 1, wherein the angle of the light source is adjustable in relation to the print bed.

8. The 3D bioprinter of claim 1, further comprising an adjustable light source holder configured to adjust the position and angle of the light source in relation to the print bed.

9. The 3D bioprinter of claim 1, further comprising a bioprinter toolhead holder configured to hold and secure the bioprinter toolhead.

10. The 3D bioprinter of claim 1, further comprising a cable management piece configured to hold and secure one or more wire or cable running to the bioprinter toolhead.

11. The 3D bioprinter of claim 1, further comprising a visible light-curable biomaterial.

12. A 3D bioprinter system, comprising:
a 3D bioprinter according to claim 1;
a visible light-curable biomaterial provided in a non-cured state and capable of being dispensed by the bioprinter toolhead; and
a source of viable cells.

13. The system of claim 12, wherein the viable cells comprise microorganisms.

14. The system of claim 12, wherein the cells are viable in the biomaterial.

15. A method for 3D printing a visible light-curable biomaterial, comprising:
providing a 3D bioprinter according to claim 1;
providing a visible light-curable biomaterial in a non-cured state;
printing a desired amount of the non-cured biomaterial; and
photocuring the printed non-cured biomaterial with visible light output from the light source.

16. The method of claim 15 wherein the biomaterial comprises viable cells.

17. The method of claim 16, wherein the viable cells comprise microorganisms.

18. The method of claim 16, further comprising culturing the cured biomaterial under appropriate growth conditions for the cells.

19. The method of claim 16, wherein the cells remain at least 25% viable 24 hours after photocuring the printed non-cured biomaterial.
